(19) United States
(12) Patent Application Publication
(10) Pub. No.: US 2013/0273519 A1
(43) Pub. Date: Oct. 17, 2013

(54) COMPOSITION FOR TREATING BLOOD AND SET OF DIAGNOSTIC KIT COMPRISING THE SAME TO DETECT AUTOIMMUNE DISEASE

(71) Applicants: In Chan Youn, Seoul (KR); Kwang Meyung Kim, Seoul (KR); Kui Won Choi, Seoul (KR); Ick Chan Kwon, Seoul (KR); Jun Uk Chu, Ochang-Eup (KR); Jong Woong Park, Seoul (KR); Soo Young Yoon, Seoul (KR); Sung Jae Choi, Seoul (KR); Ae Ju Lee, Seoul (KR); Young Mo Kang, Daegu (KR)

(72) Inventors: In Chan Youn, Seoul (KR); Kwang Meyung Kim, Seoul (KR); Kui Won Choi, Seoul (KR); Ick Chan Kwon, Seoul (KR); Jun Uk Chu, Ochang-Eup (KR); Jong Woong Park, Seoul (KR); Soo Young Yoon, Seoul (KR); Sung Jae Choi, Seoul (KR); Ae Ju Lee, Seoul (KR); Young Mo Kang, Daegu (KR)

(21) Appl. No.: 13/669,187

(22) Filed: Nov. 5, 2012

(30) Foreign Application Priority Data
Apr. 16, 2012 (KR) 10-2012-0039386

(51) Int. Cl. C12Q 1/37; C12N 5/078

(52) U.S. Cl. CPC C12Q 1/37; C12N 5/0634
USPC 435/2

(57) ABSTRACT

The present invention relates to a composition for treating blood, a set of a diagnostic kit comprising the same to detect an autoimmune disease, and a method of monitoring an autoimmune disease using the same.

An autoimmune disease such as rheumatoid arthritis can be early diagnosed, and disease progression and a treatment response can be precisely predicted, using a technique of amplifying enzyme by stimulating blood obtained from patient.

### CROSS-REFERENCE TO RELATED APPLICATION

This application claims priority from Korean Patent Application No. 10-2012-0039386, filed on Apr. 16, 2012 in the Korean Intellectual Property Office, the disclosure of which is incorporated herein by reference in its entirety.

### BACKGROUND OF THE INVENTION

1. Field of the Invention

The present invention relates to a composition for treating blood, a set of a diagnostic kit comprising the same to detect an autoimmune disease, and a method of monitoring an autoimmune disease using the same. More particularly, the present invention relates to a technique of early diagnosing rheumatoid arthritis (RA) comprising amplifying MMP by stimulating peripheral blood of a patient having RA with the composition for treating blood of the present invention, and placing the amplified matrix metalloproteinase (MMP) onto a diagnostic kit coated with optical probe complexes which specifically react with RA factors.

Further, the present invention relates to a technique of precisely and easily distinguishing a patient having RA, and monitoring the treatment response using blood sample obtained from the patient.

2. Background of the Invention

Rheumatoid arthritis is an autoimmune disease of which precise cause has not been known so far. The rheumatoid arthritis causes inflammation on the synovial joints, and destroys joints including small joints such as fingers and toes, and hip joints such as elbows, shoulders and knees. The rheumatoid arthritis is a whole body disease which results in exercise disorders and joint transformations, and which causes various types of organ damages. If early diagnosis is delayed or early intensive treatment fails, the rheumatoid arthritis changes into an irreversible chronic disease, which may cause bad results such as a patient's losing a job and shortened lifespan.

Only a rheumatoid factor (RF) among biomarkers is included in rheumatoid arthritis diagnosis criteria by the American College of Rheumatology organized in 1987, the RA diagnosis criteria currently used in clinical laboratories. However, the RF may often cause an ambiguous diagnosis, without being beneficial to early diagnosis, due to its low diagnostic specificity and sensitivity. Accordingly, there is a limitation in using the RF as a subsidiary means to clinically confirm a diagnosis.

If the rheumatoid arthritis is early treated, arthralgia or arthrokleisis can be improved. Further, if a treatment reaction is good, the quality of life can be enhanced, and problems such as radiological bone destruction, severe body disorders and life-shortening can be prevented or delayed. An erythrocyte sedimentation rate (ESR) or a C-reactive protein (CRP) currently used to monitor disease treatment effects, has a limitation in non-specifically reacting with a whole body disease or inflammation, not with a specific substance in the joint. Accordingly, the ESR or CRP has a limitation in being used as a specific examination indicator.

Examinations on antinuclear factors, anti-keratin antibodies, anti-RA 33 and anti-Sa antibodies which have been recently developed for an early diagnosis of rheumatoid arthritis in a serum manner and considered to be applicable to clinical laboratories, have higher specificity than the conventional examinations on a rheumatoid factor (RF). However, due to a low sensitivity to diseases and complicated test procedures, the examinations are not widely used in clinical laboratories. Anti-cyclic citrullinated peptide antibodies (anti-CCP), a biomarker included in rheumatoid arthritis diagnosis criteria revised in 2010 by the American College of Rheumatology, also has high specificity, but has a low sensitivity. This may cause early diagnosis not to be performed, and a treatment response not to be precisely monitored due to an unclear correlation between an anti-CCP concentration and a disease activity. In order to overcome the disadvantages, matrix metalloproteinases (MMPs) is being spotlighted as a candidate for other biomarker. Since the MMP is directly generated from the synovium, it serves as an important enzyme associated with destruction of rheumatoid arthritis. Owing to such advantages, the MMP is expected to be used for early diagnosis, and to monitor treatment effects. Accordingly, research on the correlation between the expression of the MMPs and progression of RA has been widely performed. Kits for measuring the level of expression of MMPs using an MMPs antibody have been mainly developed. Most of documents on the change of expression of MMP according to progression of RA, demonstrate non-specific results on all quantified activated and inactivated MMPs produced and secreted in the human's body. Accordingly, required is a method for quantitatively examining activated MMP which directly influences on progression of rheumatoid arthritis. There have been reported about examinations on MMPs expressed in the synovium or synovium cells of a patient with RA. However, there has been reported no research on a potential productivity of MMP in peripheral blood cells. There are documents on changes of the concentration of MMP and the level of expression of the MMP after drug treatment. However, such documents demonstrate a low consistency, and there has been no research on an prediction examination on a treatment response of RA.

In the meantime, the KR Patent No. 10-1103548 discloses a nano particle sensor for measuring the activity of matrix metalloproteinase (MMP) consisting of a fluorophore, a quencher, a peptide substrate specifically degraded by MMP, and a biocompatible polymer. This relates to a technique for imaging the level of expression of MMP in a tissue, by introducing the nano particle sensors into a patient's tissue. Thus the KR Patent No. 10-1103548 is differentiated from the technique for quantifying and imaging MMP by amplifying a very small amount of MMP in peripheral blood of a patient.

In the specification of the present invention, a plurality of theses and patent literatures were cited and referred. Through the cited theses and patent literatures, the technique of the present invention can be more clearly explained.

### SUMMARY OF THE INVENTION

The present inventors have researched on development of early diagnosis of an autoimmune disease using peripheral blood of a patient. As result of intense research, they found that matrix metalloproteinase (MMP) plays an important role in bone inflammation of early rheumatoid arthritis, and the number of macrophages in peripheral blood which produce the MMP is different between a normal person and a patient with rheumatoid arthritis. Based on such facts, they developed a method of maximizing the difference of the expression levels of matrix metalloproteinase (MMP) in macrophages between a normal person and a patient with rheumatoid arthritis, by stimulating blood with a chemical substance.

Furthermore, the present inventors have developed a molecular diagnostic kit onto which a fluorescent sensor specifically reacting with MMP has been applied. Based on the molecular diagnostic kit, they developed a technique of quantifying and monitoring the difference of the expression levels of matrix metalloproteinase (MMP) in blood of a normal person and a patient with rheumatoid arthritis, according to each chemical factor before and after stimulating the blood.

Therefore, an object of the present invention is to provide a composition for treating blood for diagnosing an autoimmune disease capable of maximizing the difference of the levels of expression of matrix metalloproteinase (MMP) in a patient's peripheral blood.

Another object of the present invention is to provide a set of a diagnostic kit for detecting an autoimmune disease, the set comprising said blood treating composition and a molecular diagnostic kit coated with fluorescent sensors.

Still another object of the present invention is to provide a method for treating blood capable of maximizing the difference of the levels of expression of matrix metalloproteinase (MMP) in a patient's peripheral blood, using the blood treating composition.

Yet still another object of the present invention is to provide a method of quantifying or imaging matrix metalloproteinase (MMP) in blood so as to provide information for diagnosis of an autoimmune disease.

To achieve these and other advantages and in accordance with the purpose of this specification, as embodied and broadly described herein, there is provided a composition for treating blood for diagnosis of an autoimmune disease, the composition including one or more blood stimulants selected from the group consisting of LPS(Lipopolysacharide), PMA(Phorbol 12-myristate 13-acetate), TNF-α(Tumor necrosis factor-alpha), IL-1β(interleukin-1β) and GM-CSF(Granulocyte-macrophage colony-stimulating factor).

The final purpose of the present research was to provide a method of identifying a rheumatoid factor from peripheral blood samples obtained from a patient with rheumatoid arthritis and a normal person, so as to enhance an early diagnosis rate of an autoimmune disease, and to enhance the activity of treatment and the accuracy of the response prediction. Various types of immune cells such as macrophage and dendritic cell are contained in blood, and the number of the immune cells increases as an immune disease progresses. The macrophages are known to produce matrix metalloproteinase (MMP), which is to be measured by the present inventor. Accordingly, the present invention is based on the concept that the difference of the levels of expression of matrix metalloproteinase (MMP) in blood of a patient with rheumatoid arthritis and a normal person can be maximized, by stimulating each blood sample obtained from the patient having rheumatoid arthritis and the normal person, with a specific chemical substance of the same concentration.

The composition of the present invention make it possible to early diagnose an autoimmune disease by maximizing the difference of the levels of expression of matrix metalloproteinase (MMP) in peripheral blood. The autoimmune disease may be osteoarthritis or rheumatoid arthritis.

The MMP is zinc- and calcium-dependent endopeptidases related to integrin signal transmittance, and a cell movement by pericellular matrix degradation, which may include without limitation at least one selected from a group consisting of MMP-1, MMP-2, MMP-3, MMP-7˜MMP-21, MMP-22, MMP-23A, MMP-23B, and MMP-24˜MMP-28.

To achieve these and other advantages and in accordance with the purpose of this specification, as embodied and broadly described herein, there is also provided a set of a diagnostic kit for detecting an autoimmune disease, comprising: (i) the composition for treating blood; and (ii) a kit coated with a complex comprising fluorophore-peptide-quencher, wherein the peptide is a peptide substrate specifically degraded by matrix metalloproteinase (MMP).

For early diagnosis of an autoimmune disease using peripheral blood, the fluorescence intensity may be monitored. Here, the fluorescence intensity is measured according to the level of expression of MMP by applying the stimulated blood onto the kit coated with optical probe complexes which specifically reacts with rheumatoid arthritis factors.

The optical probe complex which specifically reacts with rheumatoid arthritis factors may be a complex comprising fluorophore-peptide-quencher, the complex based on peptide prepared by using an amino acid sequence which is known to as a substrate of matrix metalloproteinase (MMP). If the peptide is specifically degraded by matrix metalloproteinase (MMP), the fluorophore is released from the quencher thus to express fluorescence.

In an embodiment, the fluorophore may be cyanin, fluorescein, tetramethylrhodamine, alexa or bodipy. Preferably, the fluorophore may be cyanin-based Cy 5.5(Ex/Em 670/690) which can interfere with cells, blood, body tissues, etc. to the minimum, or which can be absorbed thereto to the minimum, by emitting and absorbing double near-infrared light.

In another embodiment, the quencher may be a black hole quencher or a blackberry quencher. Generally, a quenching effect is maximized by using a quencher having a wavelength equal to or similar to that of a fluorophore. Accordingly, if Cy5.5 is used as a fluorophore, BHQ-3 (abs. 620 nm-730 nm) having a wavelength similar to that of the fluorophore may be preferably used as a quencher.

Preferably, the optical probe complex may further comprise a polymer coupled to the peptide (e.g., an MMP substrate). The use of polymer make it possible that a larger number of optical probe complexes can be fixed onto the kit easily. If a plurality of optical probe complexes are firstly bound to the polymers and then the polymer are fixed onto the kit, more optical probe complexes can be applied to the kit, compared with the case where the optical probe complexes are individually fixed onto the surface of the kit.

In an embodiment, as the polymer, may be used chitosan, dextran, hyaluronic acid, polyamino acid or heparin.

As a peptide substrate specifically degraded by the MMP enzyme, a proper substrate may be used according to a type of enzyme. For instance, for quantification of MMP-2, MMP-3, MMP-9 or MMP-13, may be used a peptide substrate including an amino acid sequence of Gly-Pro-Leu-Gly-Val-Arg-Gly-Lys-Gly-Gly. For quantification of MMP-3, MMP-7 or MMP-13, may be used a peptide substrate including an amino acid sequence of Gly-Val-Pro-Leu-Ser-Leu-Thr-Met-Gly-Lys-Gly-Gly. For quantification of MMP-2, MMP-3 or MMP-13, may be used a peptide substrate including an amino acid sequence of Gly-Pro-Leu-Gly-Met-Arg-Gly-Leu-Gly-Lys-Gly-Gly.

In case of using an in-vitro diagnostic kit onto which an optical probe complex of a peptide substrate including an amino acid sequence of Gly-Val-Pro-Leu-Ser-Leu-Thr-Met-Gly-Lys-Gly-Gly has been applied, the fluorescence intensity was high when reacting with MMP-3, MMP-7 or MMP-13 among various subgroups, and especially specificity was remarkable with respect to the MMP-3. Furthermore, the in-vitro diagnostic kit onto which an optical probe complex has been applied shows the fluorescence intensity which increases in proportion to the concentration of MMP. Accordingly, disease activity and progression of rheumatoid arthritis can be monitored by quantitatively analyzing specific MMP in blood.

To achieve these and other advantages and in accordance with the purpose of this specification, as embodied and broadly described herein, there is still also provided a method for treating blood capable of maximizing the difference of the levels of expression of matrix metalloproteinase (MMP), by stimulating blood with using at least one selected from a group consisting of LPS(Lipopolysacharide), PMA(Phorbol 12-myristate 13-acetate), TNF-α(Tumor necrosis factor), IL-1β(interleukin-1β) and GM-CSF(Granulocyte-macrophage colony-stimulating factor).

In order to quantify and/or image matrix metalloproteinase (MMP) in peripheral blood by the method for treating blood of the present invention, may be used any protein quantifying method well-known to those skilled in the art.

For instance, may be used the in-vitro diagnostic kit onto which an optical probe complex has been applied, the optical probe complex of an MMP specific peptide substrate. Alternatively, may be used a flow cytometer, or an Enzyme linked Immunosolbent assay (ELISA) currently presented on the market.

To achieve these and other advantages and in accordance with the purpose of this specification, as embodied and broadly described herein, there is yet still also provided a method for quantifying or imaging matrix metalloproteinase (MMP) in blood, the method comprising the steps of: (i) stimulating blood or amplifying components in blood by using the composition for treating blood of the present invention; and (ii) applying the treated blood to a kit coated with a complex of fluorophore-peptide-quencher.

The method for quantifying or imaging matrix metalloproteinase (MMP) exhibits a sensitivity having a similar level to a minimum concentration which can be detected by an ELISA currently presented on the market. Accordingly, the method may be preferably used to provide information on early diagnosis of an autoimmune disease. The present invention may have the following advantages.

Firstly, an autoimmune disease such as rheumatoid arthritis can be early diagnosed, and disease progression and a treatment response can be precisely predicted, through a technique for amplifying a substrate enzyme by stimulating a patient's blood cells

Secondly, owing to a simple measuring method, a disease can be effectively treated, and treating time and costs can be reduced.

Further scope of applicability of the present application will become more apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from the detailed description.

### BRIEF DESCRIPTION OF THE DRAWINGS

The accompanying drawings, which are included to provide a further understanding of the invention and are incorporated in and constitute a part of this specification, illustrate exemplary embodiments and together with the description serve to explain the principles of the invention.

In the drawings:

FIG. 1 is a mimetic diagram of an optical probe complex of fluorophore-peptide-quencher-polymer, which illustrates that a complex of fluorophore-peptide-quencher is coupled to glycol chitosan polymer;

FIG. 2 is a mimetic diagram of an in-vitro diagnostic kit which expresses fluorescence by specifically reacting with matrix metalloproteinase (MMP);

FIG. 3 is an experimental result which illustrates specificity of an in-vitro diagnostic kit with respect to MMP according to the present invention, in which the kit expressed fluorescence 40-fold by specifically reacting with MMP-3 among various MMPs, the kit onto which an optical probe complex of a peptide substrate including an amino acid sequence of Gly-Val-Pro-Leu-Ser-Leu-Thr-Met-Gly-Lys-Gly-Gly has been applied;

FIG. 4 shows the fluorescence intensity measured according to concentration of MMP-3;

FIG. 5 shows the fluorescence intensity measured according to MMP-3 in serum;

FIG. 6 shows the level of expression of MMP-3 in an animal model with rheumatoid arthritis in a quantitative manner according to weeks;

FIG. 7 is a mimetic diagram of a method for stimulating peripheral blood of a patient with rheumatoid arthritis;

FIGS. 8A and 8B show the level of expression of MMP-3 according to the number of stimuli applied to blood of a patient with rheumatoid arthritis, by using an in-vitro diagnostic kit of the present invention; and

FIG. 9 shows the level of expression of MMP-3 in blood of a patient with rheumatoid arthritis, the level of expression measured by using a flow cytometer.

### DETAILED DESCRIPTION OF THE INVENTION

Description will now be given in detail of the exemplary embodiments, with reference to the accompanying drawings. The embodiments and advantages are merely exemplary and are not to be construed as limiting the present disclosure. This description is intended to be illustrative, and not to limit the scope of the claims.

### EXAMPLES

### Example 1

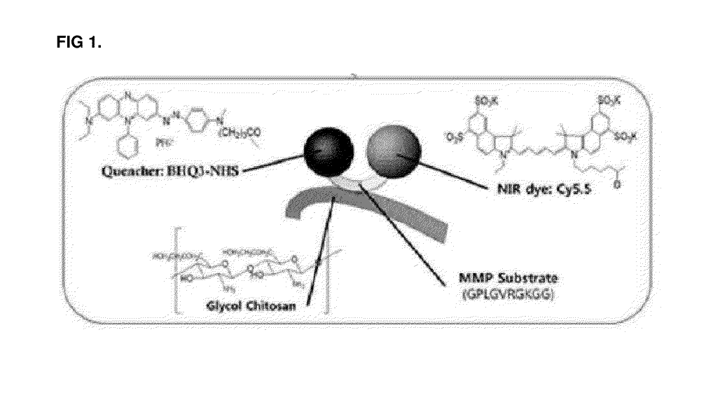

### Preparation of Complex of Fluorophore-Peptide-Quencher-Polymer (FIG. 1)

To provide a complex of fluorophore-peptide-quencher, a peptide was firstly prepared by Fmoc solid phase synthesis. Then, a fluorophore and a quencher were chemically coupled to the prepared peptide.

More specifically, Cy5.5 (ex/em, 670/690) was used as the fluorophore, and BHQ-3 (abs. 620 nm-730 nm) was used as the quencher for quenching the Cy5.5. As a peptide substrate specifically degraded by MMP, NH₂-Gly-Val-Pro-Leu-Ser-Leu-Thr-Met-Gly-Lys(Boc)-Gly-Gly-COOH was prepared by Fmoc peptide synthesis method. Then, 8.5 mg of Cy5.5-HNS ester, a near-infrared fluorophore, 8 μl of N-methylmorpholine, and 0.3 mg of 4-dimethylaminopyridine were dissolved in 200 μl of dimethylformamide. Then, the solution was reacted with 5 mg of the peptide substrate at room temperature for 12 hours. The resultant was precipitated in 4 ml of cold ethyl ether, and centrifuged. The supernatant was removed, and the remnant was washed again with 2 ml of cold ethyl ether. Ethyl ether above the surface of the remnant was removed, and the remaining substance was dried using a speed vacuum or a vacuum oven to obtain a peptide precursor, Cy5.5-Gly-Val-Pro-Leu-Ser-Leu-Thr-Met-Gly-Lys(Boc)-Gly-Gly-COOH.

In order to remove the protection group of the dried peptide precursor, the substance was reacted with 1 ml of trifluoroacetic acid, 25 μl of distilled water and 25 μl of anisole, at room temperature for 1 hour. The solvent was completely removed using a rotary pump, and the remaining substance was dissolved in 1 ml of HPLC eluent (saline solution including 0.1% TFA:acetonitrile including 0.1% TFA=1:1). Then, the solution was filtered out using a filter (0.45 pm, applicable to an organic solution). The HPLC was stabilized in 5% acetonitrile including 0.1% TFA and 95% saline solution including 0.1% TFA, using an HPLC eluent (saline solution including 0.1% TFA:acetonitrile including 0.1% TFA=1:1) and an agilent ZORBAX SB-C18 column (9.4×150 mm). Substance separation was performed for 20min, through a gradient elution (5% for 0 min, 22% for 5 min, 40% for 20 min, acetonitrile including 0.1% TFA vs DW including 0.1% TFA). After measuring absorbance at 220 nm (UV), 675 nm (FLD ex) and 690 nm (em), Cy5.5-Gly-Val-Pro-Leu-Ser-Leu-Thr-Met-Gly-Lys-Gly-Gly-COOH was isolated. A molecular weight of the isolated substance was measured by mass spectrometry, and the substance was freeze-dried. 2 mg of the substance was reacted at room temperature for 12 hours, with a solution where BHQ3-NHS ester (Biosearch Technologies Inc., 0.71 mg), 1.5 μl of NMM, and 0.2 mg of DMAP are dissolved in 30 μl of DMSO. Then, the HPLC was stabilized in 5% acetonitrile including 0.1% TFA and 95% saline solution including 0.1% TFA, by using an HPLC eluent (saline solution including 0.1% TFA:acetonitrile including 0.1% TFA=1:1) and an agilent ZORBAX SB-C18 column (9.4×150 mm). Substance separation was performed, through a gradient elution, for 25 min (5% for 0 min, 30% for 5 min, 70% for 25 min, acetonitrile including 0.1% TFA vs saline solution including 0.1% TFA). After measuring absorbance at 220 nm (UV), 675 nm (FLD ex) and 690 nm (em), Cy5.5-Gly-Val-Pro-Leu-Ser-Leu-Thr-Met-Gly-Lys(BHQ3)-Gly-Gly-COOH was isolated. A molecular weight of the isolated substance was measured by mass spectrometry, and the substance was freeze-dried.

Then, polymers were used in order to fix a large amount of complexes of fluorophore-peptide-quencher onto a 24-well plate. It is more efficient to fix a plurality of complexes of fluorophore-peptide-quencher to polymers and then fix the polymers onto a 24-well plate, rather than to fix a plurality complexes of fluorophore-peptide-quencher onto a 24-well plate directly. As the polymer, used was glycol chitosan having biocompatibility and a molecular weight of 250,000 Da.

The prepared complex of fluorophore-peptide-quencher was dissolved in 100 μl DMSO. To the solution, 100 μl of PBS (pH 6.0) was added and then 1 mg of EDC and 0.8 mg of NHS were added for reaction at room temperature for 15 min. Then, the solution was added to a solution where 10mg of glycol chitosan is dissolved in 15 ml of PBS (pH 7.4), and reacted at room temperature for 12 hours. Then, the fluorophore-peptide-quencher not having been reacted for 3 days was removed by dialysis.

A mimetic diagram of the prepared complex of fluorophore-peptide-quencher was shown in FIG. 1.

### Example 2

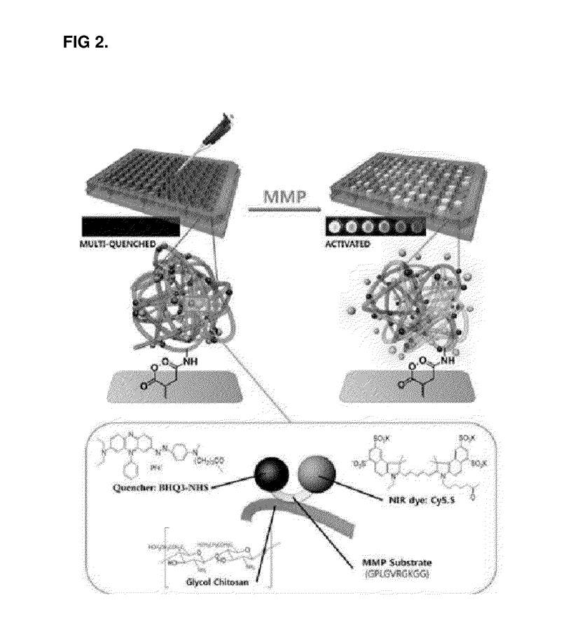

### Preparation of In-Vitro Diagnostic Kit Expressing Fluorescence by Specifically Reacting with MMP (FIG. 2)

The complex of fluorophore-peptide-quencher-polymer prepared in the Example 1 was applied onto a kit having amine (—NH3) attached thereto, and reacted at room temperature for 12 hours. As a result, prepared was an in-vitro kit having amine onto which the complex of fluorophore-peptide-quencher-polymer is chemically coupled, the kit configured to express fluorescence by specifically reacting with MMP.

### Example 3

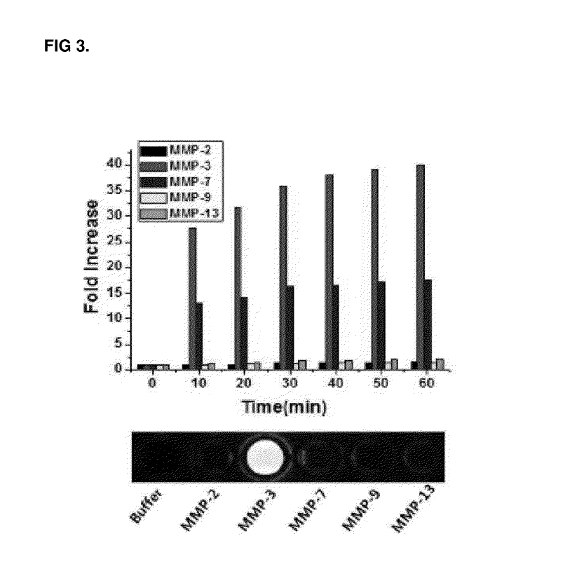

### Observation of Specificity of In-Vitro Diagnostic Kit with Respect to MMPS, the Kit Coated with Complex of Fluorophore-Peptide-Quencher-Polymer (FIG. 3)

In order to observe the specificity of the in-vitro kit prepared in the Example 2 with respect to MMP, commercially available MMP-2, MMP-3, MMP-7, MMP-9 and MMP-13 (R&D systems) were prepared, and activated with TCNB reaction solution (0.1 M Tris, 5 mM calcium chloride, 200 mM NaCl, 0.1% Brij) containing p-aminophenyl mercuric acid (SIGMA). The MMPs were reacted with the TCNB reaction solution at 37 for 1 hour. Each activated MMP was put into the in-vitro kit prepared in the Example 2 instead of serum, and the fluorescence intensity thereof was observed. The fluorescence intensity with respect to each MMP was measured, using an F-7000 fluorescence spectrophotometer manufactured by Hitachi, at 675 nm (ex) and 676˜800 nm (em).

When the kit was reacted with MMP-3, 40 times or more intense fluorescence is observed (FIG. 3). From such observation, it was confirmed that the in-vitro kit of the present invention can be used to measure the level of MMP-3 in blood of a patient with rheumatoid arthritis.

### Example 4

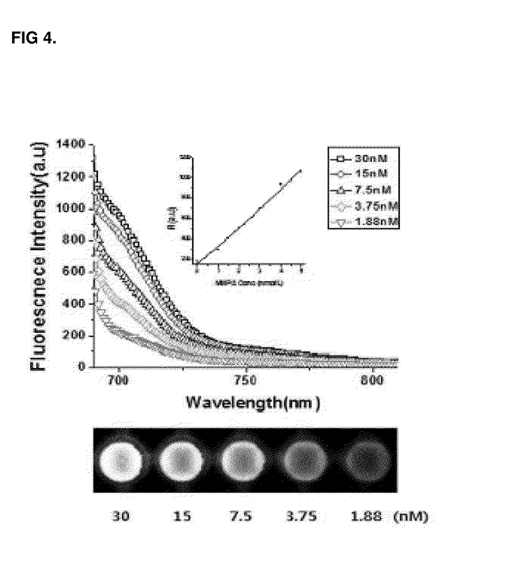

### Fluorescence Intensity of Complex According to Concentration of MMP, and Imaging MMP (FIG. 4)

The dependency of the diagnostic kit prepared in the Example 2 on MMP-3 concentration was observed. In the same manner as described in the Example 3, 1.88. 3.75, 7.5, 15 and 30 nM of the activated MMP-3 were added to the diagnostic kit respectively, and the fluorescence intensity was measured using a fluorescence spectrophotometer.

As a result, obtained was a linear graph having a value of R²=0.991 dependent on the MMP-3 concentration (FIG. 4). Through the linear graph, it was indirectly proven that the fluorescence intensity is variable according to the level of expression of MMP in blood.

### Example 5

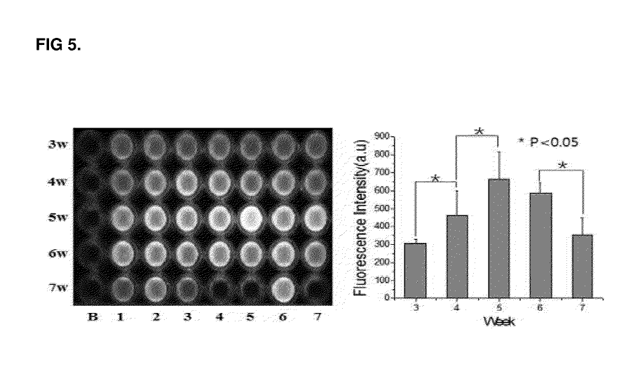

### Measuring Fluorescence Intensity by MMP-3 in Serum (FIG. 5)

Male DBA/1J mice, 5 weeks of age, were used as a rheumatoid arthritis model. The mixture of type II collagen, immunity-reinforcing agent (adjuvant) and H37RA bacteria was subcutaneously injected into the tails of the mice slowly. After 2 weeks, the same procedure was performed again to boost the effects.

Mice models of rheumatoid arthritis were prepared as described above, and the serum samples were obtained from 7 mice according to weeks (3 weeks, 4 weeks, 5 weeks, 6 weeks and 7 weeks).

More specifically, LPS (SIGMA) having a concentration of 100 ng/ml was added to the medium to stimulate the blood, and the DBA/1J mouse's blood obtained according to weeks was added to the medium in a 10-fold diluted state. Then, the blood was stirred well not to form a lump, and incubated in a cell incubator under the condition of 5% CO₂ and 37. After 3 hours, the blood mixed with the medium was transferred into a 2 ml tube, and centrifuged under the condition of 3500 rpm/4/5 min to isolate the supernatant. The serum specimen prepared as above was applied onto the diagnostic kit prepared in the Example 2, so as to observe the intensity of fluorescence using a fluorescence spectrophotometer.

The highest level of MMP-3 expression was observed in the mice of 5 weeks, which is in the negligible stage of rheumatoid arthritis (hardly visible to the naked eye) between the first and second stage (total 4 stages) on the list of marks for rheumatoid arthritis. Therefore, it is confirmed that the fluorescence intensity is increased depending on the MMP-3 concentration (FIG. 5).

### Example 6

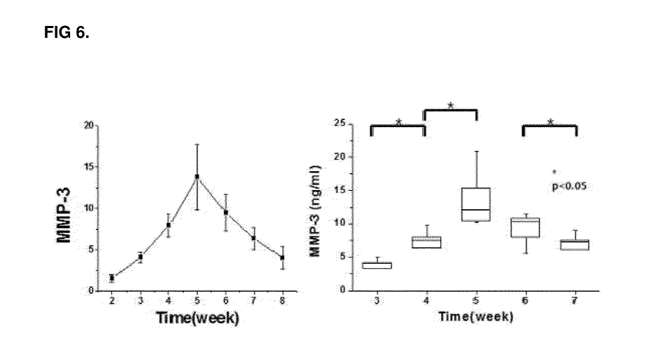

### Quantitative Measurement of the Expression Level of MMP in Animal Model With Rheumatoid Arthritis (FIG. 6)

The expression level of MMP was determined based on the concentration of recombinant human MMP-3 in the standard specimen used in the diagnostic kit of the present invention. Furthermore, the expression level of MMP in the same specimen was determined using the ELISA technique (Enzyme-Linked ImmunoSorbent Assay), which is the method used worldwide to quantify a protein using an antibody response. From the results, it was proven that the sensitivity of the diagnostic kit of the present invention is similar to that of ELISA using an antibody, and the level of expression of MMP was statistically valid.

### Example 7

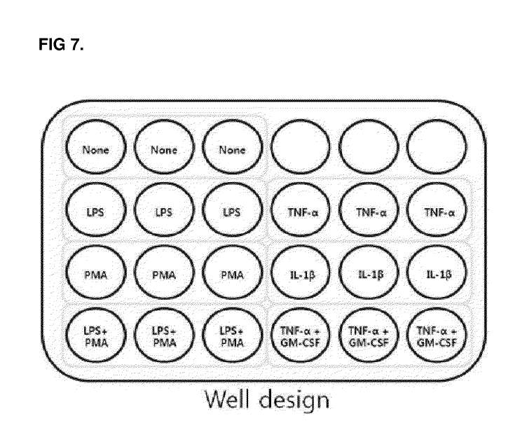

### Stimulating Peripheral Blood of Patient and Normal Person (FIG. 7)

The specimens were collected at the same time from a patient and a normal person, and immediately processed. RPMI 1640(GIBCO) was used as a medium for cell culture for blood stimulation. No antibiotic and no FBS were added to the blood to prevent any influences on cell amplification since the blood is stimulated just for a short time. To stimulate the blood cells, used were LPS(Lipopolysacharide), PMA(Phorbol 12-myristate 13-acetate)(SIGMA), TNF-α(Tumor necrosis factor)(R&D Systems), IL-1β(interleukin-1β)(Calbiochem) and GM-CSF (Granulocyte-macrophage colony-stimulating factor)(R&D Systems). A proper combination of the substances was added to the medium, and the concentration of each substance used was as follows.

| Substance | Concentration (ng/ml) |
|---|---|
| LPS | 100 |
| PMA | 50 |
| LPS + PMA | 100 + 50 |
| TNF-a | 50 |
| IL-1B | 50 |
| TNF-a + GM-CSF | 50 + 25 |

The above substances were respectively added to the 50 ml medium, and stored in a refrigerator. The substances was warmed in 37 water prior to use. A 24-well plate which can contain total 1 ml volume of medium was used for cell culture. In the present experimentation, the blood was diluted 10 times with medium, and transferred into a tube of which surface is coated with heparin. After 900 ul of medium was added to the each well, the blood was well mixed with the medium shaking up and down in order to prevent serum and plasma from being separated from each other. Then, 100 ul of the blood was put into the each well containing medium. The pipette tip was continuously changed into a new one to prevent contamination. After adding blood, the 24-well plate was put onto an agitator, shaken well not to form a lump, and transferred into a cell incubator under the condition of 5% CO₂ and 37. The present inventors performed experiments three times with respect to each medium (N=3) to reduce deviation. After 3 hours, the blood mixed with the medium was transferred into a 2 ml tube, and centrifuged under the condition of 3500 rpm/4/5 min. The supernatant of the centrifuged blood was isolated and stored in a freezer of −80.

A mimetic diagram of wells into which respective culture mediums were applied for stimulus of peripheral blood, was shown in FIG. 7.

### Example 8

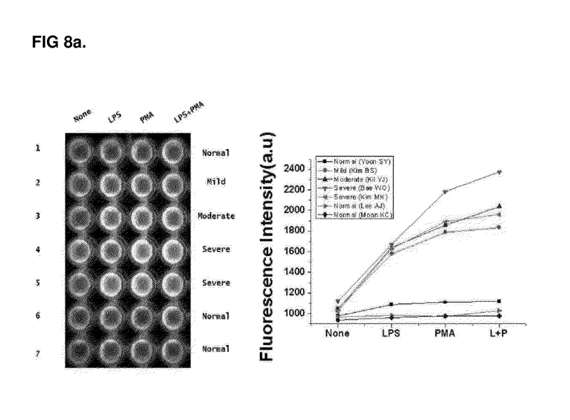

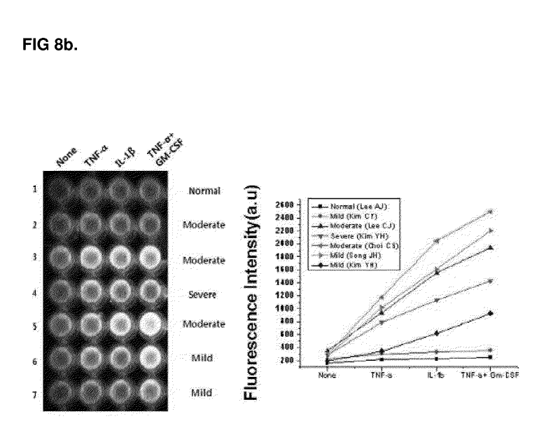

### Test on Efficiency of Diagnostic Kit Using the Sample of Embodiment 7 (FIG. 8)

In order to measure the amount of MMP amplified in blood of a patient with rheumatoid arthritis and a health person prepared in the Example 7, the tube stored at −80 was taken out, and melted slowly in an ice-water bath. 200 ul of the blood was extracted using pipette, and put into a 1.5 ml tube. Then, the tube was kept warm in 37 water for 1 hour. In addition, for the samples to be used as positive controls in the kit, recombinant human MMP-3 was diluted ×50, ×100, ×200, ×400, or ×800 times, and kept warm in 37 water for 15 min. Then, APMA (aminophenylmercuric acetate) was added to the blood to activate MMP-3, and left alone for 1 hour. During the time, 0.5% bovine serum albumin was added to the kit of which surface is coated with MMP-specific nano-probes, and placed at room temperature for 1 hour, stirring to prevent nonspecific reaction. After 1 hour, 150 ul of the blood sample was put into the kit, and placed at 37 for 8 hours. Since the sample inside the kit may be evaporated by temperature, sides of the kits were completely sealed. After lapse of 8 hours, the intensity of fluorescence inside the kit was measured as a numerical value using an optical imaging apparatus. Then, an imaging process was performed with respect to the fluorescence, and the captured image was compared with the patient's information for data processing.

It was observed that the fluorescence intensity increased in the specimen of the patient with severe rheumatoid arthritis than in the normal person's specimen (FIG. 8). From such results, it is anticipated that rheumatoid arthritis can be early diagnosed, and disease progression can be monitored instantly.

### Example 9

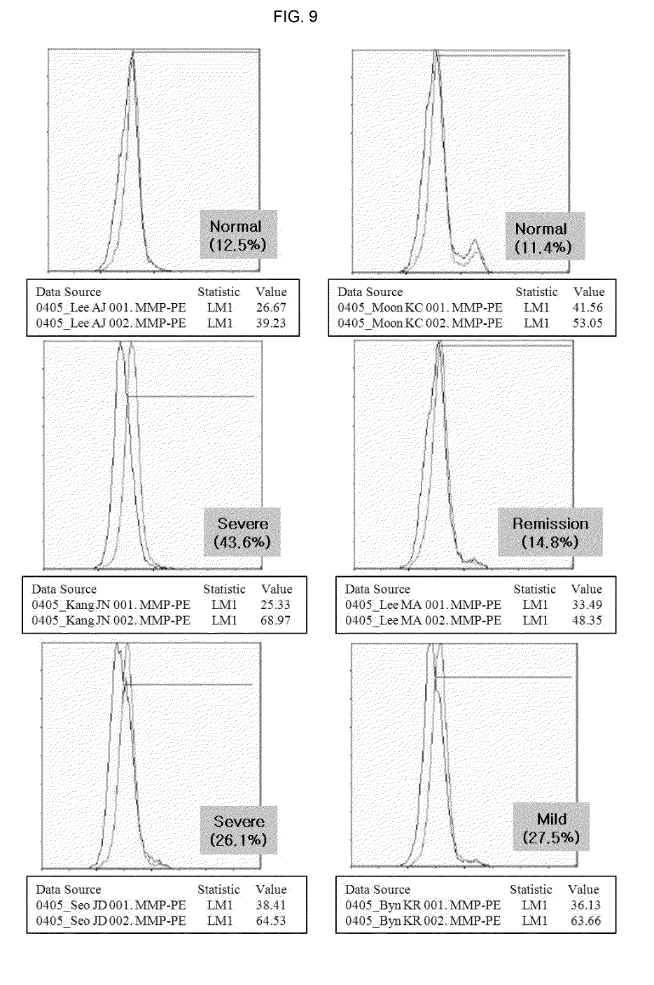

### Measuring the Level of Expression of MMP-3 in Blood Using Flow Cytometer (FIG. 9)

In order to implement the method of stimulating blood and to check the clinical value of the kit of the present invention, the level of expression of MMP-3 was checked using a flow cytometer. 3 ml of blood was collected to sodium citrate tube (BD vacutainer), and CBC (complete blood count) was measured immediately upon pumping-up the blood. Then, the blood was stimulated with the chemical substance of the present invention. 500 ul of whole blood was put into the 24 wells plate for cell culture, and 90 ng of PMA was added into the plate. Then, the cell culture plate was put into a 5% CO₂ incubator, and was kept at 37 for 3 hours. After 3 hours, 200 ug/ml of Brefeldin A(SIGMA) was added into the plate, and placed at 37 for 6 hours.

After lapse of the total 9 hours, 50 ul of blood was extracted, and put into 2 tubes respectively. Then, 10 ul of CD45-PC5 10 and 10 ul of CD14-FITC were respectively added to the 2 tubes, mixed with each other, and placed in a darkroom at room temperature for 15 min. Then, 100 ul of RBC lysis buffer (Bechman coulter) was added to the 2 tubes respectively, and mixed with each other using a vortex. The mixtures were placed in a darkroom at room temperature for 15 min. Then, 4 ml of PBS was added to the 2 tubes respectively, and centrifuged to remove the supernatant. Here, the supernatant was not removed using pipette, but poured out to minimize the loss of cells. 100 ul of 0.1% saponin was added to the remaining cells, and stirred smoothly using pipette. Then, the mixture was placed in a darkroom at room temperature for 5min. Then, 10 ul of IgG1-PE was added to the control group and 10 ul of MMP-3-PE was added to the comparative group, and then the both groups were placed in a darkroom for 15 min. After 15 min, 4 ml of PBS was added to the each group, and centrifuged under the condition of 2000 g, 5 min and 4 to remove the supernatant. Then, 500 ul of fresh PBS was added to the each group, and fluorescence-activated cell sorting (FACS) was performed. The results of the FACS analysis were shown in FIG. 9.

Data shown in FIG. 9 is summarized as in the table below.

| | Normal1 | Normal2 | RA (S) | RA (R) | RA (s) | RA (mi) |
|---|---|---|---|---|---|---|
| Before | 26.67 | 41.56 | 25.33 | 33.49 | 38.41 | 36.13 |
| After | 39.23 | 53.05 | 68.97 | 48.35 | 64.53 | 63.65 |
| Total | 12.56 | 11.49 | 43.64 | 14.86 | 26.12 | 27.52 |

Normal: Normal person
RA (S): Patient with severe rheumatoid arthritis
RA (R): Patient with reduced activity after being treated with drugs
RA (mi): Patient with mild rheumatoid arthritis

From the experimental results, it was observed that the level of expression of MMP-3 increased in the specimen of the patient with severe rheumatoid arthritis than in the normal person's specimen (FIG. 8). Such results were consistent with the results obtained in the Example 7 using the in-vitro diagnostic kit of the present invention.

The foregoing embodiments and advantages are merely exemplary and are not to be considered as limiting the present invention. The present teachings can be readily applied to other types of apparatuses. This description is intended to be illustrative, and not to limit the scope of the claims. Many alternatives, modifications, and variations will be apparent to those skilled in the art. The features, structures, methods, and other characteristics of the exemplary embodiments described herein may be combined in various ways to obtain additional and/or alternative exemplary embodiments.

As the present features may be embodied in several forms without departing from the characteristics thereof, it should also be understood that the above-described embodiments are not limited by any of the details of the foregoing description, unless otherwise specified, but rather should be considered broadly within its scope as defined in the appended claims, and therefore all changes and modifications that fall within the metes and bounds of the claims, or equivalents of such metes and bounds are therefore intended to be embraced by the appended claims.

1. A method of treating blood to maximize the difference of expression levels of matrix metalloproteinase (MMP) in blood samples, comprising the step of stimulating blood with the composition comprising one or more blood stimulants selected from the group consisting of LPS (Lipopolysacharide), PMA (Phorbol 12-myristate 13-acetate), TNF-α (Tumor necrosis factor-alpha), IL-1β (interleukin-1β) and GM-CSF (Granulocyte-macrophage colony-stimulating factor).

2. A method of quantifying or imaging matrix metalloproteinase (MMP) in blood to diagnose an autoimmune disease, the method comprising the steps of:
(i) treating blood using the method of claim 1; and
(ii) applying the treated blood to a kit coated with a complex comprising a conjugate of fluorophore-peptide-quencher,
wherein the peptide is a peptide substrate specifically degraded by matrix metalloproteinase (MMP).

3. A method of quantifying matrix metalloproteinase (MMP) in blood so as to diagnose an autoimmune disease, the method comprising the steps of:
(i) treating blood using the method of claim 1; and
(ii) quantifying matrix metalloproteinase (MMP) in the treated blood, using a flow cytometer.

4. The method of claim 2, wherein the autoimmune disease is ostarthritis or rheumatoid arthritis.

5. The method of claim 2, wherein the fluorophore is selected from the group consisting of cyanin, fluorescein, tetramethylrhodamine, alexa and bodipy.

6. The method of claim 2, wherein the quencher is a black hole quencher or a blackberry quencher.

7. The method of claim 2, wherein the complex further comprises a polymer coupled to the peptide.

8. The method of claim 7, wherein the polymer is selected from the group consisting of chitosan, dextran, hyaluronic acid, polyamino acid and heparin.

9. The method of claim 3, wherein the autoimmune disease is ostarthritis or rheumatoid arthritis.


SEQUENCE LISTING (7 sequences)

SEQ ID NO: 1 - protein, 10 aa, Artificial Sequence
  Description of Artificial Sequence Synthetic peptide
GPLGVRGKGG

SEQ ID NO: 2 - protein, 12 aa, Artificial Sequence
  Description of Artificial Sequence Synthetic peptide
GVPLSLTMGKGG

SEQ ID NO: 3 - protein, 12 aa, Artificial Sequence
  Description of Artificial Sequence Synthetic peptide
GPLGMRGLGKGG

SEQ ID NO: 4 - protein, 12 aa, Artificial Sequence
  Description of Artificial Sequence Synthetic peptide
GVPLSLTMGKGG

SEQ ID NO: 5 - protein, 12 aa, Artificial Sequence
  Description of Artificial Sequence Synthetic peptide
GVPLSLTMGKGG

SEQ ID NO: 6 - protein, 12 aa, Artificial Sequence
  Description of Artificial Sequence Synthetic peptide
GVPLSLTMGKGG

SEQ ID NO: 7 - protein, 12 aa, Artificial Sequence
  Description of Artificial Sequence Synthetic peptide
GVPLSLTMGKGG